(19) United States
(12) Patent Application Publication
(10) Pub. No.: US 2013/0338201 A1
(43) Pub. Date: Dec. 19, 2013

(54) Method of Cancer Treatment with 2-(1H-Indole-3-Carbonyl)-Thiazole-4-Carboxylic Acid Methyl Ester

(71) Applicant: AhR Pharmaceuticals, Inc., Madison, WI (US)

(72) Inventor: Jiasheng Song, Madison, WI (US)

(73) Assignee: AhR Pharmaceuticals, Inc., Madison, WI (US)

(21) Appl. No.: 13/954,834

(22) Filed: Jul. 30, 2013

Related U.S. Application Data
(63) Continuation-in-part of application No. 13/503,657, filed on Apr. 24, 2012.
(60) Provisional application No. 61/257,422, filed on Nov. 2, 2009.

(51) Int. Cl. A61K 31/427; A61K 45/06; C07D 417/06

(52) U.S. Cl. CPC A61K 31/427; C07D 417/06; A61K 45/06
USPC 514/365

(57) ABSTRACT

A method of cancer treatment is provided that includes administering an effective amount of an endogenous ligand for the aryl hydrocarbon (Ah) receptor (AhR) named ITE or one of its structural analogs (the active ingredient) to a subject with cancer is disclosed. An effective dose and dosing frequency of the active ingredient are determined by measuring its blood levels of the subject after dosing. The active ingredient formulated with a carrier system is applied topically, enterally, or parenterally to the subject. An oral dose of water, in addition to normal water drinking, is administered to help alleviate feces hardening, a complication of ITE dosing. Subjects with cancers of skin, colon (or rectum), stomach, pancreas, kidney, bladder, soft tissue, and cervix, are preferably accepted for treatment or intervention.

### RELATED APPLICATIONS

This application is a continuation-in-part application of, and claims priority of, U.S. application Ser. No. 13/503,657, now allowed, filed Apr. 24, 2012, which claims priority to International Application No. PCT/US2010/052729, filed Oct. 14, 2010, which claims priority to U.S. provisional application 61/257,422, filed Nov. 2, 2009, now abandoned, the entire disclosures of which are incorporated herein by this reference in their entirety.

### TECHNICAL FIELD

The presently disclosed invention relates to a method of cancer treatment using 2-(1′H-indole-3′-carbonyl)-thiazole-4-carboxylic acid methyl ester (“ITE”) or one of its structural analogs. More particularly, the present invention provides a method by administering a therapeutically effective amount of ITE or one of its structural analogs to treat cancer in a subject in need thereof including skin, colorectal, stomach, pancreatic, kidney, bladder, soft tissue, and cervical cancer.

### BACKGROUND

The aryl hydrocarbon (Ah) receptor (AhR) is a ligand inducible transcription factor, a member of the basic helix-loop-helix/Per-Arnt-Sim (bHLH/PAS) superfamily. Upon binding to its ligand, AhR mediates or interacts with a series of biological processes as well as some adverse effects including cell division, apoptosis, cell differentiation, actions of estrogen and androgen, adipose differentiation, hypothalamus actions, angiogenesis, immune system homeostasis, teratogenicity, tumorigenicity, chloracne, wasting syndrome, and actions of other hormonal systems beside the expression of genes of P450 family and others. The liganded receptor participates in biological processes through translocation from cytoplasm into nucleus, heterodimerization with another factor named Ah receptor nuclear translocator, attachment of the heterodimer to the regulatory region termed Ah response element of genes under AhR regulation, and then either enhancement or inhibition of transcription of those genes.

The AhR happens to be able to bind, with different affinities, to several groups of exogenous chemicals (thus artificial ligands) such as polycyclic aromatic hydrocarbons exemplified by 3-methylchoranthrene (3-MC) and halogenated aromatic hydrocarbons typified by 2,3,7,8-tetrachlorodibenzo-p-dioxin (TCDD). The receptor system has been studied so far with its artificial ligands. While these studies helped greatly in advancing our understanding toward the receptor system, thorough elucidation of the physiological roles the system plays and the potential therapeutic benefits the system may offer are impossible without the identification of AhR physiological ligand. As the first step toward this goal, an endogenous ligand for the receptor has been identified. The endogenous ligand, or physiological ligand, or natural hormone, for the AhR was identified as 2-1′H-indole-3′-carbonyl)-thiazole-4-carboxylic acid methyl ester (short for ITE).

Even though most of the artificial ligands for AhR are environmental toxins and thus cannot be used as therapeutic agents, for the purpose of understanding functions of liganded AhR, its artificial ligands such as TCDD, 6-methyl-1,3,8-trichlorodibenzofuran (6-MCDF), 8-methyl-1,3,6-trichlorodibenzofuran (8-MCDF), and those derived from indole or tryptophan were used to reveal that the liganded AhR was able to inhibit the metastasis of prostate tumors in a strain of transgenic mice and the growth of carcinogen induced rat mammary tumors, human breast tumor cell xenografts, and tumors caused by gene mutations.

As a natural ligand for AhR, ITE is an excellent agent in targeting precisely and specifically the receptor. The consequence of the targeting, however, is unpredictable from the behaviors of those artificial ligands for AhR, with some results demonstrating anticancer potentials while others tumor initiation, promotion, and progression. As disclosed in a U.S. patent application Ser. No. 13/503,657, it is impossible to predict meaningfully what a newly identified ligand for AhR might do in terms of cancer biology. The same application also explained that, as an antiangiogenic agent, ITE would not be automatically qualified as an effective anticancer agent either.

In the area of cancer treatment, however, there are two serious problems with current cancer therapies in the market. The first is side effects and toxicity. The second is efficacy. Consequently, cancer is still the second leading cause of death in the United States and areas of the world.

The majority of current therapeutic agents for cancer, in both cytotoxic and noncytotoxic categories, are chemicals foreign to the human body. As a result, the body tries to reject the agents using metabolic methods available. Since the human body does not have a natural and safe way of metabolizing those foreign chemicals, some nonspecific oxidation reactions then are used as major means of metabolism. The consequence is that the metabolic processes generate chemically active intermediates or radicals, which will assault also normal cellular substances including, but not limited to, that of immune system's in the body, leading to side effects, toxicity, and weakened immune system. Since most of these agents were designed by humans, not the nature, they have very high chances to bind to and interact with other cellular factors (including, but not limited to, receptors, enzymes, other proteins) than their expected targets in the body. These “off-target” bindings and interactions account for opportunities for side effects.

The effectiveness of cytotoxic agents for cancer therapy is mainly limited by their indiscriminate toxicity to normal cells and tissues including, but not limited to, that of immune system's. The weakened immune system makes it impossible to launch an organized assault on cancer cells. The efficacy of noncytotoxic agents, which target specific functions important for the survival of cancer cells, is limited by their single mechanism based strategy. An important hallmark of cancer, however, is their constant genetic changes or mutations. Once a cancer cell changes into a state that it is no longer dependent on a specific function a therapeutic agent targets for survival, the efficacy of the agent will then be lost.

The situation thus calls for the emergence of a novel therapeutic agent that can assault cancers cells with multiple combating capabilities for sustained potency, help immune system at the same time to organize an orchestrated attack on cancers and clean up individual cancer cells for a possible cancer eradication, and limit the chance of “off-target” interaction and metabolize itself safely for low side effect(s). In addition, feces hardening seems to be a complication of ITE dosing, which may render the passage of food and feces through intestinal system difficult. That may complicate crucial life processes and then lead to occasional death of ITE dosed animals in the most severe cases.

Therefore, there is an unmet medical need to develop a method of cancer treatment with fewer side-effects and higher efficacy, in addition to find methods to protect normal cells from injuries caused by cancer treatment. Further, it is also desirable to development methods of cancer treatment that can alleviate the complication as disclosed herein.

### SUMMARY OF THE INVENTION

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

Further advantages of the presently-disclosed subject matter will become evident to those of ordinary skill in the art after a study of the description, Figures, and non-limiting Examples in this document.

The presently disclosed invention provides a method of cancer treatment including administering a therapeutically effective amount of 2-(1′H-indole-3′-carbonyl)-thiazole-4-carboxylic acid methyl ester or one of its structural analogs. In some embodiments, the cancer is selected from a group consisting of skin, colorectal, stomach, pancreatic, kidney, bladder, soft tissue, and cervical cancer. The structure analog of 2-(1′H-indole-3′-carbonyl)-thiazole-4-carboxylic acid methyl ester has the following formula:

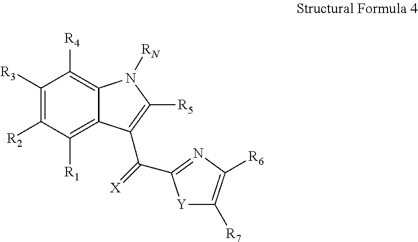

wherein
X and Y, independently, can be either O (oxygen) or S (sulfur);
R_{N} can be selected from hydrogen, halo, cyano, formyl, alkyl, haloalkyl, alkenyl, alkynyl, alkanoyl, haloalkanoyl, or a nitrogen protective group;
R₁, R₂, R₃, R₄, and R₅ can be independently selected from hydrogen, halo, hydroxy (—OH), thiol (—SH), cyano (—CN), formyl (—CHO), alkyl, haloalkyl, alkenyl, alkynyl, amino, nitro (—NO₂), alkoxy, haloalkoxy, thioalkoxy, alkanoyl, haloalkanoyl, or carbonyloxy;
R₆ and R₇, can be independently selected from hydrogen, halo, hydroxy, thiol, cyano, formyl, alkyl, haloalkyl, alkenyl, alkynyl, amino, nitro, alkoxy, haloalkoxy, or thioalkoxy; or
R₆ and R₇, independently, can be:

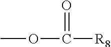

wherein R_{g} can be selected from hydrogen, halo, cyano, alkyl, haloalkyl, alkenyl, or alkynyl; or R₆ and R₇, independently, can be:

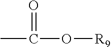

wherein R₉ can be selected from hydrogen, halo, alkyl, haloalkyl, alkenyl, or alkynyl; or R₆ and R₇, independently, can be:

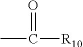

wherein R₁₀ can be selected from hydrogen, halo, hydroxy, thiol, cyano, alkyl, haloalkyl, alkenyl, alkynyl, amino, or nitro; or
R₆ and R₇, independently, can also be:

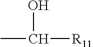

wherein R₁₁ can be selected from hydrogen, halo, alkyl, haloalkyl, alkenyl, or alkynyl.

In some embodiments, the structure of the 2-(1′H-indole-3′-carbonyl)-thiazole-4-carboxylic acid methyl ester is represented by the following structural formula:

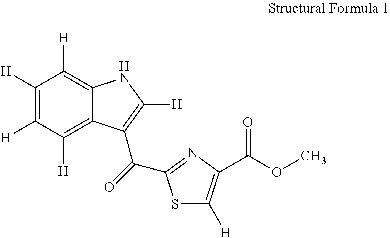 [SMILES: COC(=O)c1csc(C(=O)c2c[nH]c3ccccc23)n1]

In some further embodiments, the structural analog of 2-(1′H-indole-3′-carbonyl)-thiazole-4-carboxylic acid methyl ester is represented by the following structural formula:

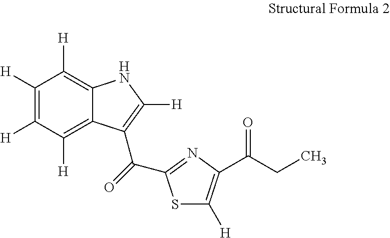 [SMILES: CCC(=O)c1csc(C(=O)c2c[nH]c3ccccc23)n1]

Yet in some embodiments, the structural analog of 2-(1′H-indole-3′-carbonyl)-thiazole-4-carboxylic acid methyl ester is represented by the following structural formula:

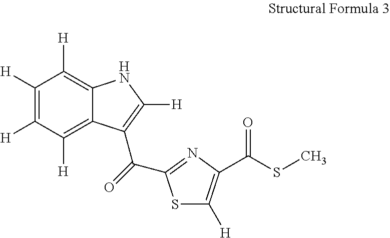 [SMILES: CSC(=O)c1csc(C(=O)c2c[nH]c3ccccc23)n1]

Further provided, in some embodiments, the 2-(1′H-indole-3′-carbonyl)-thiazole-4-carboxylic acid methyl ester or one of its structural analogs is combined with one or more pharmaceutically acceptable carriers to assist its administration to the subject. Yet in some embodiments, the 2-(1′H-indole-3′-carbonyl)-thiazole-4-carboxylic acid methyl ester or one of its structural analogs can be administered using topical, enteral, and parenteral application.

Still further provides, in some embodiments, the presently disclosed invention relates to a method that a dose of water is administered to the subject to reduce complication of dosing 2-(1′H-indole-3′-carbonyl)-thiazole-4-carboxylic acid methyl ester or one of its structural analogs. In some embodiments, the dose of water administered is in excess to normal water uptake by the subject. Yet in some embodiments, the dose of water is administered by oral injection.

In some embodiments of the presently disclosed invention, the 2-1′H-indole-3′-carbonyl)-thiazole-4-carboxylic acid methyl ester or one of its structural analogs is administered together with one or more other cancer therapeutic agents to the subject.

In some embodiments of the presently disclosed invention, a maintenance dosing of the 2-1′H-indole-3′-carbonyl)-thiazole-4-carboxylic acid methyl ester or one of its structural analogs is provided after the subject is free of cancer to ensure cancer eradication.

In some embodiments, the subject in need of treatment for cancer is a mammal. Yet in some embodiments, the mammal is a human.

Further provided, in some embodiments of the present invention, a subject is administered with the 2-(1′H-indole-3′-carbonyl)-thiazole-4-carboxylic acid methyl ester or one of its structural analogs once or twice daily.

Advantages of the presently-disclosed subject matter will become evident to those of ordinary skill in the art after a study of the description, Figures, and non-limiting Examples in this document.

### BRIEF DESCRIPTION OF DRAWINGS

FIG. 1 are graphs illustrating the growth inhibition (mean±SEM, n=5) of cancer tissues derived from human and mouse cancer cells in response to ITE administration (i.p. injection, DMSO as vehicle, once daily continuous) at doses of 100-200 mg/kg (100 mg/kg first 10 days and 200 mg/kg then) or 200-300 mg/kg (200 mg/kg first 6 days and 300 mg/kg then) as indicated, wherein FIG. 1A shows the growth inhibition of A375 (skin cancer) xenografts (100-200 mg/kg), and wherein FIG. 1B shows the growth inhibition of M14 (skin cancer) xenografts (100-200 mg/kg), and wherein FIG. 1C shows the growth inhibition of HCT116 (colon cancer) xenografts (100-200 mg/kg), and wherein FIG. 1D shows the growth inhibition of SW116 (colon cancer) xenografts (100-200 mg/kg), and wherein FIG. 1E shows the growth inhibition of SGC7901 (stomach cancer) xenografts (100-200 mg/kg), and wherein FIG. 1F shows the growth inhibition of MFC (stomach cancer) homografts (200-300 mg/kg).

FIG. 2 are graphs illustrating the growth inhibition (mean±SEM, n=5) of cancer tissues derived from human and mouse cancer cells by ITE administration (i.p. injection, DMSO as vehicle, once daily continuous) at doses of 100-200 mg/kg (100 mg/kg first 10 days and 200 mg/kg then) or 200 mg/kg as indicated, wherein FIG. 2A shows the growth inhibition of CFPAC-1 (pancreatic cancer) xenografts (100-200 mg/kg), and wherein FIG. 2B shows the growth inhibition of Ketr-3 (kidney cancer) xenografts (100-200 mg/kg), and wherein FIG. 2C shows the growth inhibition of T24 (bladder cancer) xenografts (100-200 mg/kg), and wherein FIG. 2D shows the growth inhibition of S180 (soft tissue cancer or sarcoma) homografts (200 mg/kg), and wherein FIG. 2E shows the growth inhibition of Siha (cervical cancer) xenografts (100-200 mg/kg), and wherein FIG. 2F shows the growth inhibition of U14 (cervical cancer) homografts (200 mg/kg).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth herein to facilitate explanation of the presently-disclosed subject matter. Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

The most important advantage of the present invention is ITE's or one of its structural analogs' multiple cancer assaulting capabilities to defy the consequence of cancer cells' constant genetic changes.

As used herein, the term “Ah receptor” or “AhR” stands for a receptor named aryl hydrocarbon receptor, a ligand inducible transcription factor in biological systems. In literature, the Ah receptor (AhR) liganded with its artificial ligands was shown to be able to inhibit cell division, promote apoptosis, induce cell differentiation, and block actions of estrogen and androgen. Recently, AhR liganded with artificial ligands has been demonstrated to be able to induce the differentiation of immune T cells, useful for the immune system in organizing assault on pathogens and cancers. If ITE, or one of its structural analogs, when bound to AhR, can also have one or more of the functions mentioned plus its antiangiogenic capability, the multiple cancer assaulting capabilities may make its cancer therapeutic potency sustainable. The sustainability of the potency of ITE or one of its analogs plus its potential capability of stimulating the immune system to conduct precision attack on and individual clearing of cancer cells would not only enhance dramatically the efficacy of the cancer therapy but also make cancer eradication a possibility.

A benefit of using ITE over others in the market is its possibility of low or no severe side effect(s) beside its sustainable efficacy backed by its multiple cancer assaulting capabilities. Contrary to those chemicals, including those AhR artificial ligands and most of agents used in current cancer therapies, foreign to human body and designed by humans, ITE is a natural hormone designed by the nature and so the nature may have designed and implemented a natural and safe way for its metabolism. Its metabolic process thus will cause less or even no problem to the body. This means that it may be low in side effect(s) caused by its metabolism. Another important reason for possible low side effect is that the binding of the natural hormone to its receptor (AhR) is supposed to be very specific and precise since it is designed by the nature, not humans. The natural hormone ITE, other than those human designed chemicals, will then have low chance of binding to and interacting with other cellular factors thus reducing or even eliminating “off-target” actions, important opportunities for side effects.

Another important issue in cancer therapy is that it is highly desirable for a therapeutic agent specifically working in cancer cells vs. normal cells to further enhance its potency and reduce side effects. This type of specificity can be achieved if there are more target molecules the agent binds in cancer cells than in normal cells. The target molecule for ITE and its analogs is AhR. In literature, AhR was reported to be highly concentrated in pancreatic cancer tissues from patients but very diluted in all normal pancreatic tissues examined. Similarly, the concentrated AhR is also documented with cancers of prostate, stomach, ovary, liver, lung, and esophagus. This means that the therapeutic specificity of ITE and its structural analogs could be achieved in these reported types of cancers at least.

The presently disclosed invention relates to a method of cancer treatment using 2-(1′H-indole-3′-carbonyl)-thiazole-4-carboxylic acid methyl ester (“ITE”) or one of its structural analogs. More particularly, the present invention provides a method by administering a therapeutically effective amount of ITE or one of its structural analogs to treat cancer in a subject in need thereof including skin, colorectal, stomach, pancreatic, kidney, bladder, soft tissue, and cervical cancer.

As used herein, the term “structural analog” or simply “analog” of ITE is defined as a compound with chemical structure similar to that of Ah receptor endogenous ligand ITE and with a capability of binding to the Ah receptor.

The term “treatment” or “treating” refers to curing or substantially curing a condition, as well as ameliorating at least one symptom of the condition, and are inclusive of prophylactic treatment and therapeutic treatment. As would be recognized by one of ordinary skill in the art, treatment that is administered prior to clinical manifestation of a condition then the treatment is prophylactic (i.e., it protects the subject against developing the condition). If the treatment is administered after manifestation of the condition, the treatment is therapeutic (i.e., it is intended to diminish, ameliorate, control, or maintain the existing condition and/or side effects associated with the condition). In some embodiments of the present invention, the terms further relate to cancer intervention or eradication, which means an action of reducing, inhibiting, or eliminating the illness or a pathological process or processes.

The term “therapeutically effective amount” is used herein to refer to an amount of the therapeutic agents (e.g., ITE or its structural analogs) sufficient to produce a measurable biological response (e.g., cancer intervention or eradication). Actual dosage levels of active ingredients in a therapeutic composition of the presently disclosed subject matter can be varied so as to administer an amount of the active compound(s) that is effective to achieve the desired therapeutic response for a particular subject and/or application. The selected dosage level will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and dose is escalated in the absence of dose-limiting toxicity to a minimally effective amount. Determination and adjustment of a therapeutically effective dose, as well as evaluation of when and how to make such adjustments, are known to those of ordinary skill in the art of medicine.

The terms “subject,” as used herein, refer to an individual to be treated by (e.g., administered) methods of the present invention. Subjects include, but are not limited to, mammals (e.g., murines, simians, equines, bovines, porcines, canines, felines, and the like), and most preferably includes humans. In the context of the invention, the term “subject” generally refers to an individual who will be administered or who has been administered one or more compound/compositions of the present invention (e.g., a composition for cancer treatment).

ITE or one of its analogs (the active ingredient) can be formulated with one or more pharmaceutically acceptable carrier.

As used herein, the term “pharmaceutically acceptable carrier” refers to inert materials useful for administering the active ingredient, preferably sterile and nontoxic. The carrier system should be compatible with the active ingredient and can be in a form of solid, liquid, or gas. The properly formulated active ingredient can then be administered topically, enterally, or parenterally to a subject with cancer. It can be provided, for example, in a form of cream, capsules, tablets, lozenges, or injectables. Other compatible ingredients such as preservatives, if needed, could be co-formulated with the active ingredient. It further refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

In some embodiments of the present invention, subjects with cancers of skin, colon (or rectum), stomach, pancreas, kidney, bladder, soft tissue, and cervix are preferably accepted for treatment with ITE or one of its structural analogs. This is by no mean to limit the therapeutic scope, however. Given the multiple cancer assaulting capabilities that ITE and one of its analogs possesses plus the possibility of stimulation of a subject's immune system to precisely attack and clean up individual cancer cells for possible cancer eradication, the spectrum of the ITE therapy is envisioned to be further expanded.

Further provided, in some embodiments of the present invention, the effective dose range of ITE or one of its structural analogs is determined by measuring the subject's blood concentration of ITE or one of its structural analogs under a specified dosing regimen to establish a concentration-time profile, consulting with an established correlation between the similar concentration-time profiles and effects on cancer inhibition or eradication, which built during a trial or trials as that illustrated in the section of Examples, and balancing the therapeutic effects achievable with the possible toxicity to the subject and health condition or physical durability of the subject. The dosing frequency of ITE or one of its structural analogs is decided similarly as described for the determination of a dose range above. Currently, once a day administration either enterally or parenterally is proposed as preferable with ITE. The dosing will be continued until the subject is free from the cancer. It is preferable to provide a maintenance dosing, whose duration is directed by a trial or trials, after the subject is free of cancer to ensure its complete elimination or eradication.

In the preferred intervention program, extra water is administered orally, in addition to normal daily water drinking, to alleviate an ITE dosing complication due probably to the hardening of feces. A daily dose of 20 ml/kg of water administered orally in a short period of time is proposed preliminarily. An optimal dose level and schedule are determined by a trial or trials as illustrated in the section of Examples.

The term “complication” as used herein refers to a symptom associated with a disease or with a treatment process of a disease with effective amount of the therapeutic agents. A non-limiting example is dry or hardened feces in a subject in need of cancer treatment after administering effective amount of ITE or its analogs.

In some embodiments of the presently-disclosed invention, ITE or one of its structural analogs may be administered in combination with one or more of other cancer therapeutic agents, preferably aiming different therapeutic targets other than AhR. ITE or one of its structural analogs can be formulated either independently from or together with one or more of the other said agents. ITE or one of its structural analogs can be administered either at the same schedule with or different from that of one or more of the other said agents. The proportioning of ITE or one of its structural analogs to one or more of the other cancer therapeutic agents can be directed by a well-designed trial or trials. Combining the therapy of ITE or one of its analogs with one or more of the other cancer therapeutic agents, may further enhance the efficacy. There are lots of examples to show the benefits of combination therapy.

In some embodiments of the present invention, the active ingredient is the aryl hydrocarbon (Ah) receptor (AhR) endogenous ligand ITE with the following structural formula (Structural Formula 1):

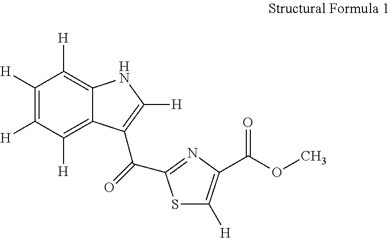 [SMILES: COC(=O)c1csc(C(=O)c2c[nH]c3ccccc23)n1]

In some other embodiments, the active ingredient can be selected from two especially useful structural analogs of ITE. The said two analogs are envisioned to increase their stability and then extend their half-life in the subjects' systems since either a ketone or thiol ester functional group replaces the normal (oxygen) ester, targeted easily by numerous esterases in biological systems, in the structure of ITE. The extended half-life may translate into higher efficacy and/or longer duration of potency in cancer intervention. The ketone analog (thus termed ITK) of ITE is of the following structural formula (Structural Formula 2):

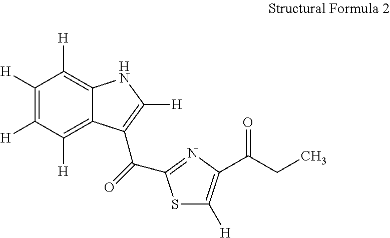 [SMILES: CCC(=O)c1csc(C(=O)c2c[nH]c3ccccc23)n1]

Whereas the structural formula of the thiol (S, sulfur) ester analog (thus termed ITSE) of ITE is as follows (Structural Formula 3):

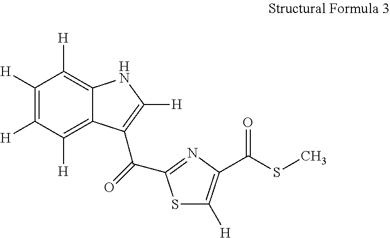 [SMILES: CSC(=O)c1csc(C(=O)c2c[nH]c3ccccc23)n1]

In some embodiments, the active ingredient can be further selected from the other structural analogs of ITE, specified by the following structural formula (Structural Formula 4):

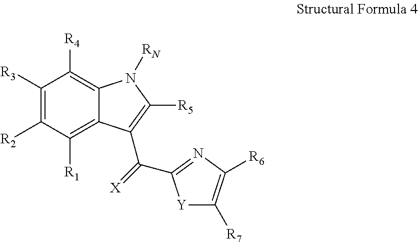

wherein
X and Y, independently, can be either O (oxygen) or S (sulfur);
R_{N} can be selected from hydrogen, halo, cyano, formyl, alkyl, haloalkyl, alkenyl, alkynyl, alkanoyl, haloalkanoyl, or a nitrogen protective group;
R₁, R₂, R₃, R₄, and R₅ can be independently selected from hydrogen, halo, hydroxy (—OH), thiol (—SH), cyano (—CN), formyl (—CHO), alkyl, haloalkyl, alkenyl, alkynyl, amino, nitro (—NO₂), alkoxy, haloalkoxy, thioalkoxy, alkanoyl, haloalkanoyl, or carbonyloxy;
R₆ and R₇, can be independently selected from hydrogen, halo, hydroxy, thiol, cyano, formyl, alkyl, haloalkyl, alkenyl, alkynyl, amino, nitro, alkoxy, haloalkoxy, or thioalkoxy; or
R₆ and R₇, independently, can be:

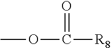

wherein R₈ can be selected from hydrogen, halo, cyano, alkyl, haloalkyl, alkenyl, or alkyn yl; or R₆ and R₇, independently, can be:

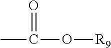

wherein R₉ can be selected from hydrogen, halo, alkyl, haloalkyl, alkenyl, or alkynyl; or R₆ and R₇, independently, can be:

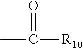

wherein R₁₀ can be selected from hydrogen, halo, hydroxy, thiol, cyano, alkyl, haloalkyl, alkenyl, alkynyl, amino, or nitro; or
R₆ and R₇, independently, can also be:

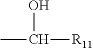

wherein R₁₁ can be selected from hydrogen, halo, alkyl, haloalkyl, alkenyl, or alkynyl.

As used herein, the term “alkyl” represents a group of hydrogen saturated one to six carbons connected in either straight or branched fashion.

As used herein, the term “haloalkyl” represents an alkyl substituted by one or more halogen atoms.

As used herein, the term “alkenyl” represents a group of hydrocarbons containing two to six carbons connected in either straight or branched fashion with at least one carbon-to-carbon double bond.

As used herein, the term “alkynyl” represents a group of hydrocarbons containing two to six carbons connected in either straight or branched fashion with at least one carbon-to-carbon triple bond.

As used herein, the term “halo” represents any of halogen atoms (F, Cl, Br, or I).

As used herein, the term “carbonyl” represents:

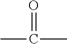

As used herein, the term “alkanoyl” represents an alkyl connected to a carbonyl group:

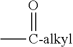

As used herein, the term “haloalkanoyl” represents a haloalkyl connected to a carbonyl group:

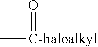

As used herein, the term “nitrogen protective group” represents groups commonly used to protect nitrogen from undesired chemical reactions during synthesis procedures.

As used herein, the term “amino” represents —NRaRb where Ra and Rb can be independently selected from hydrogen, halo, formyl (—CHO), alkyl, haloalkyl, alkenyl, alkynyl, alkanoyl, haloalkanoyl, or a nitrogen protective group.

As used herein, the term “alkoxy” represents an alkyl connected to an oxygen atom (—O-alkyl).

As used herein, the term “haloalkoxy” represents a haloalkyl connected to an oxygen atom (—O-haloalkyl)

As used herein, the term “thioalkoxy” represents an alkyl connected to a sulfur atom (—S-alkyl).

As used herein, the term “carbonyloxy” represents an alkanoyl connected to an oxygen atom:

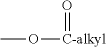

The presently-disclosed subject matter is further illustrated by the following specific but non-limiting examples. Some of the following examples are prophetic, notwithstanding the numerical values, results and/or data referred to and contained in the examples.

### EXAMPLES

Examples from animal studies will further help the embodiment of the present invention. Use of orally administered water, in addition to normal water drinking, to alleviate ITE dosing complication and use of ITE in inhibiting growth of cancers originated from varieties of organs in both human and mouse will be demonstrated.

Materials

Female BALB/c nude mice were supplied by Shanghai SLAC Laboratory Animals, Co. Ltd. (Shanghai, China). Female ICR mice were purchased from Shanghai Sippr-BK Laboratory Animal Co. Ltd. (Shanghai, China). Female 615 mice were from Institute of Hematology, Chinese Academy of Medical Sciences (Tianjin, China). Mice of 6 to 8 weeks of age were individually marked and coded. The animals were kept in laminar flow rooms at a constant temperature of 20 to 25° C. and humidity of 40 to 70%. The bedding material was corn cob, which was changed twice weekly. Animals had free access to sterile dry granule food and sterile drinking water during the entire procedure of studies.

Cell lines HCT116 (human colon cancer), M14 (human skin cancer), A375 (human skin cancer), SGC7901 (human stomach cancer), Ketr-3 (human kidney cancer), S180 (mouse soft tissue cancer/sarcoma), and U14 (mouse cervical cancer) were purchased from Chinese Academy of Medical Sciences & Peking Union Medical College (Beijing, China). Cell lines SW116 (human colon cancer), Siha (human cervical cancer), T24 (Human bladder cancer), CFPAC-1 (human pancreatic cancer), and MFC (mouse stomach cancer) were from Cell Center, Shanghai Academy of Life Sciences, Chinese Academy of Sciences (Shanghai, China).

ITE was customer manufactured by Shanghai ChemPartner Co., Ltd. (Shanghai, China) for AhR Pharmaceuticals, Inc. (Madison, Wis., USA). The lot number of the compound in AhR Pharmaceuticals' system is AHR-003. DMSO was supplied by Sigma-Aldrich (St. Louis, Mo., USA). Water used was the triple distilled water.

Methods

All the procedures related to animal handling, care, and the treatment in the study were performed following guidelines of KeyGen Biotechn Co., Ltd. (Nanjing, China), a contract research organization we hired, based on the guidance of the Association for Assessment and Accreditation of Laboratory Animal Care (AAALAC). Animals were checked for any effects of tumor growth and drug treatment on normal behavior such as mobility, food and water consumption, body weight gain/loss (bodyweights were measured every two days), eye/hair matting, and any other abnormal effect. Death and observed clinical signs were recorded. Individual animals with a tumor volume exceeding 3000 mm³ or animals of a group with a mean tumor volume exceeding 2,000 mm³ were euthanized. In addition, animals showing signs of severe distress and/or pain or losing the capability of accessing adequate food or water were humanely sacrificed.

Oral injection of water was initiated at the same day i.p. administration of ITE was started. Daily oral gavage at 10 or 20 ml/kg was performed immediately following daily i.p. injection of ITE or DMSO continuously for the entire duration of the study. Both oral and i.p. injection were accidentally skipped at day 14 due to a mistake of the experimenter(s) and all of the other dosing were executed normally. Bodyweights (individual), food consumed (in group), feces weights (in group), and feces appearance (in group) were recorded every two days. Death of animal(s) and approximate cause of death etc. were also recorded.

Cell lines HCT116, Siha, MFC were cultured in DMEM medium (high glucose) supplemented with 10% fetal bovine serum (FBS), SW116 and T24 in RPMI-1640 medium with 10% FBS, M14 and A375 in DMEM medium (high glucose) with 10% calf serum (CS), SGC7901 and Ketr-3 in RPMI-1640 medium with 10% CS, and CFPAC-1 in IMDM medium with 10% FBS. All of the cells were cultured, in the presence of 100 U/ml penicillin and 100 μg/ml streptomycin, at 37° C. in an atmosphere of 5% CO₂ in air. The tumor cells were routinely subcultured twice a week. The cells growing in an exponential phase were harvested and counted for tumor inoculation. Cell lines 5180 and U14 were maintained in ascites (peritoneal cavity fluid) of mice and were harvested and counted for tumor inoculation.

Each mouse was inoculated subcutaneously in right armpit with about 1×10⁶ cells in 0.1 ml of PBS for tumor development. While cells of human in origin were inoculated into female BALB/c nude mice, MFC cells were inoculated into female 615 mice and U14 and S180 cells into female ICR mice. When a mean tumor volume reached 50 to 150 mm³ as indicated, the tumor-bearing mice were divided randomly into vehicle control and ITE treatment group. Each group was consisted of 5 tumor-bearing mice. Vehicle (DMSO) or ITE in the vehicle at specified doses were administered to the mice by i.p. (intraperitoneal) injection at a volume of 2 ml/kg once daily for 28 continuous days or less for those reaching set tumor volumes earlier. A dose of 100-200 mg/kg designates a regimen of 100 mg/kg administered at the first 10 days and 200 mg/kg rest of days. Similarly, a dose of 200-300 mg/kg represents a scheme of 200 mg/kg dosed at the first 6 days and 300 mg/kg rest of days. Following each i.p. injection of ITE or vehicle alone, an oral gavage of 20 ml/kg water was performed to each mouse.

Tumor volume was measured every two days in two dimensions using a caliper and the volume was calculated with a formula of: V=0.5 a×b², where a and b are the long and short diameter (in mm) of a tumor, respectively. The tumor volume was then used for calculations of both TGI (Tumor Growth Inhibition) and TGD (Tumor Growth Delay). The TGI was determined by: TGI=(1−ΔT/ΔC)×100%, where ΔT was a difference between the mean tumor volume at a specified day of observation and that at the day treatment starts (day 0) for a drug treated group whereas ΔC was the same difference measured for the control group. The TGD was calculated as: TGD=T−C, where T was the time (in days) required for tumors in a drug treated group to reach a predetermined mean tumor volume and C the time (in days) in the control group to reach the same volume.

Summary statistics, including mean and the standard error of the mean (SEM), were provided for the tumor volume of each group at each time point. Statistical analysis (Student's t-Test) of difference in tumor volumes between vehicle control and ITE treatment group was conducted by Excel 2010 at each time point and P<0.05 was considered to be statistically significant.

Results

Mice administered with ITE at either low (200 mg/kg) or high (300 mg/kg) level showed “dry” feces visually after one week of dosing (Table 1). In day 28 of ITE treatment, feces of oral water treated mice became “moderate” from “dry” and kept the appearance while ITE treated mice without oral water dosing kept their feces in “dry” appearance. While most of the mice survived the entire experimental procedure for a total of 40 days, one ITE low dose (200 mg/kg) and one ITE high dose (300 mg/kg) treated mice without oral water administration did die at day 28 and 36, respectively, illustrating the potential benefit of the oral water regimen (in addition to free access to drinking water) in ITE dosed mice (Table 1). Oral gavage of 20 ml/kg water following ITE or vehicle dosing becomes a standard protective procedure in studies reported in the present invention.

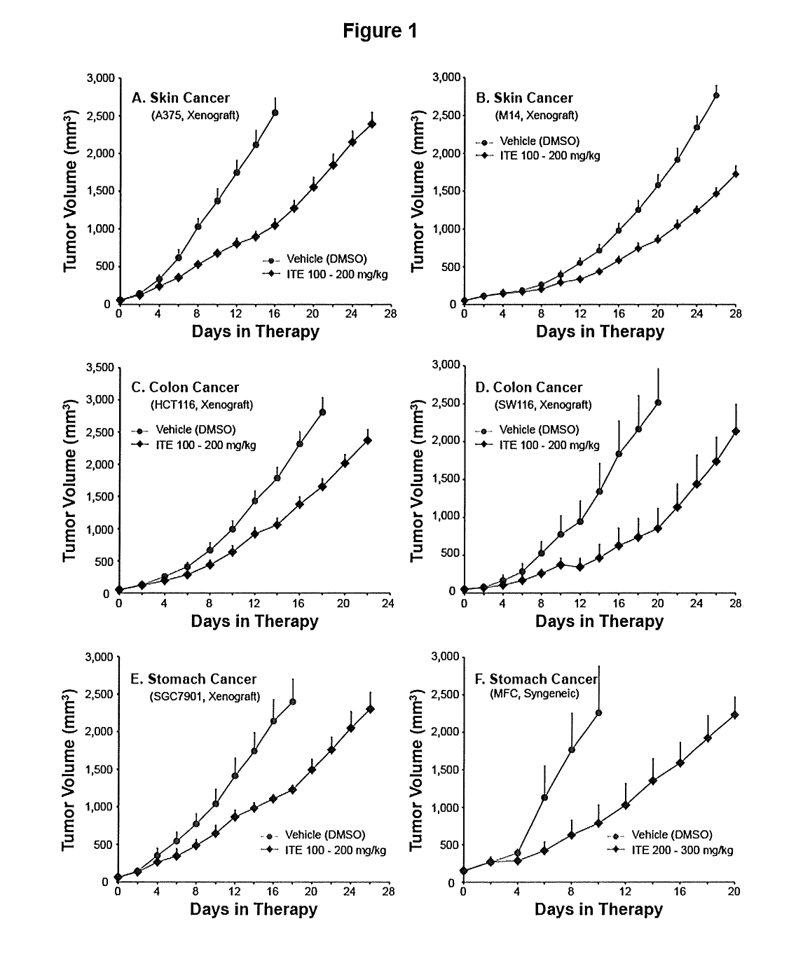

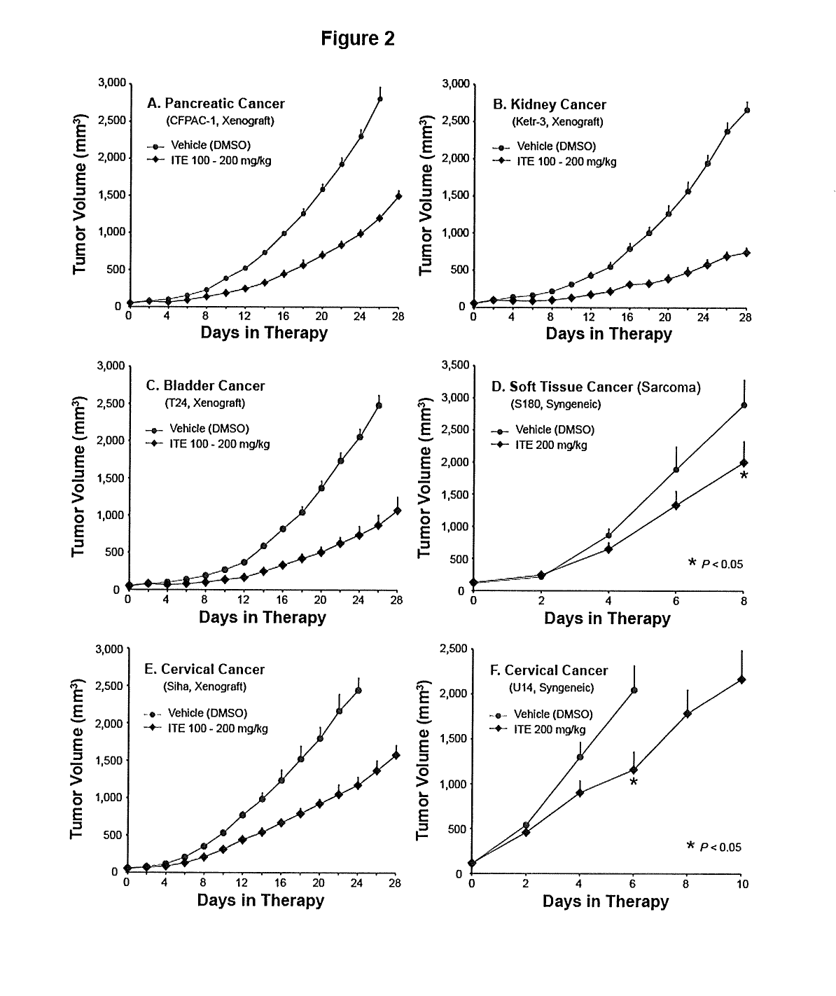

ITE demonstrated significant anticancer capability on human skin cancer xenograft model (cell line A375, FIG. 1A; cell line M14, FIG. 1B; 100-200 mg/kg), human colon cancer xenograft model (cell line HCT116, FIG. 1C; cell line SW116, FIG. 1D; 100-200 mg/kg), human stomach cancer xenograft model (cell line SGC7801, FIG. 1E; 100-200 mg/kg), mouse stomach cancer homograft (syngeneic) model (cell line MFC, FIG. 1F; 200-300 mg/kg), human pancreatic cancer xenograft model (cell line CFPAC-1, FIG. 2A; 100-200 mg/kg), human kidney cancer xenograft model (cell line Ketr-3, FIG. 2B; 100-200 mg/kg), human bladder cancer xenograft model (cell line T24, FIG. 2C; 100-200 mg/kg), mouse soft tissue cancer/sarcoma homograft (syngeneic) model (cell line S180, FIG. 2D; 200 mg/kg), human cervical cancer xenograft model (cell line Siha, FIG. 2E; 100-200 mg/kg), and mouse cervical cancer homograft (syngeneic) model (cell line U14, FIG. 2F; 200 mg/kg).

Table 1 Illustrates effects of p.o. water administration (oral gavage) on i.p. ITE dosed mice, wherein “ITE-0”, “ITE-L”, and “ITE-H” represent, respectively, no ITE, low ITE (200 mg/kg), and high ITE (300 mg/kg) treatment; and wherein “Water-0”, “Water-L”, and “Water-H” indicate, respectively, no water, low water (10 ml/kg), and high water (20 ml/kg) administration; and wherein “M” and “D” stands for “Moderate” and “Dry”, respectively, in visual inspection of feces appearance; and wherein the numbers follow either “M” or “D” represent number(s) of mice that died during experimentation.

TABLE 1
Effects of Oral Water Administration on ITE Dosed Mice

| | ITE-0 + | ITE-L + | ITE-L + | ITE-L + | ITE-H + | ITE-H + | ITE-H + |
|---|---|---|---|---|---|---|---|
| Days | Water-0 | Water-0 | Water-L | Water-H | Water-0 | Water-L | Water-H |
| 0 | M, 0 | M, 0 | M, 0 | M, 0 | M, 0 | M, 0 | M, 0 |
| 2 | M, 0 | M, 0 | M, 0 | M, 0 | M, 0 | M, 0 | M, 0 |
| 4 | M, 0 | M, 0 | M, 0 | M, 0 | M, 0 | M, 0 | M, 0 |
| 6 | M, 0 | M, 0 | M, 0 | M, 0 | M, 0 | M, 0 | M, 0 |
| 8 | M, 0 | D, 0 | D, 0 | D, 0 | D, 0 | D, 0 | D, 0 |
| 10 | M, 0 | D, 0 | D, 0 | D, 0 | D, 0 | D, 0 | D, 0 |
| 12 | M, 0 | D, 0 | D, 0 | D, 0 | D, 0 | D, 0 | D, 0 |
| 14 | M, 0 | D, 0 | D, 0 | D, 0 | D, 0 | D, 0 | D, 0 |
| 16 | M, 0 | D, 0 | D, 0 | D, 0 | D, 0 | D, 0 | D, 0 |
| 18 | M, 0 | D, 0 | D, 0 | D, 0 | D, 0 | D, 0 | D, 0 |
| 20 | M, 0 | D, 0 | D, 0 | D, 0 | D, 0 | D, 0 | D, 0 |
| 22 | M, 0 | D, 0 | D, 0 | D, 0 | D, 0 | D, 0 | D, 0 |
| 24 | M, 0 | D, 0 | D, 0 | D, 0 | D, 0 | D, 0 | D, 0 |
| 26 | M, 0 | D, 0 | D, 0 | D, 0 | D, 0 | D, 0 | D, 0 |
| 28 | M, 0 | D, 1 | M, 0 | M, 0 | D, 0 | M, 0 | M, 0 |
| 30 | M, 0 | D, 1 | M, 0 | M, 0 | D, 0 | M, 0 | M, 0 |
| 32 | M, 0 | D, 1 | M, 0 | M, 0 | D, 0 | M, 0 | M, 0 |
| 34 | M, 0 | D, 1 | M, 0 | M, 0 | D, 0 | M, 0 | M, 0 |
| 36 | M, 0 | D, 1 | M, 0 | M, 0 | D, 1 | M, 0 | M, 0 |
| 38 | M, 0 | D, 1 | M, 0 | M, 0 | D, 1 | M, 0 | M, 0 |
| 40 | M, 0 | D, 1 | M, 0 | M, 0 | D, 1 | M, 0 | M, 0 |

TGI's (Tumor Growth Inhibition), P values of Student's t-Test for the difference between vehicle and ITE treatment group in tumor volume calculated at the days TGI's were calculated, TGD's (Tumor Growth Delay), and percentages of bodyweight loss at the end of therapies (relative to the weights at the beginning) were listed in Table 2. There was no single animal death in either vehicle or ITE treatment group in any model in the entire therapies.

Table 2 lists several calculated numbers that help to further dissect the information from mouse cancer model studies, wherein “TGI (%)” means percentage of tumor growth inhibition as defined in the section of Methods, and wherein “TGI at day” represents the day at which the TGI was calculated, and wherein “P at TGI day” denotes the P value of the Student's t-Test for the difference between vehicle and ITE treatment group in tumor volume calculated at the day TGI was calculated, and wherein “TGD” indicates tumor growth delay in days as described in the section of Methods, and wherein “TGD at Vol.” stands for tumor volume at which the TGD was calculated, and wherein “Weight Loss” designates percentage of bodyweight loss at the end of the therapy relative to the weight at the beginning of the therapy.

TABLE 2
Mouse Cancer Model Studies

| Cell Line | Cancer | TGI (%) | TGI at day | P at TGI day | TGD | TGD at Vol. | Weight Loss |
|---|---|---|---|---|---|---|---|
| A375 | Skin | 60 | 16 | 1 × 10⁻⁴ | 7 | 1,000 | 18 |
| M14 | Skin | 48 | 26 | 1 × 10⁻⁴ | 5 | 1,000 | 15 |
| HCT116 | Colon | 41 | 18 | 2 × 10⁻³ | 3 | 1,000 | 9 |
| SW116 | Colon | 67 | 20 | 1 × 10⁻⁴ | 9 | 1,000 | 15 |
| SGC7901 | Stomach | 50 | 18 | 3 × 10⁻⁵ | 4 | 1,000 | 12 |
| MFC | Stomach | 70 | 10 | 1.1 × 10⁻³ | 9 | 2,000 | 16 |
| CFPAC-1 | Pancreas | 58 | 26 | 1 × 10⁻⁴ | 8 | 1,000 | 14 |
| Ketr-3 | Kidney | 74 | 28 | 2 × 10⁻⁷ | 9 | 500 | 13 |
| T24 | Bladder | 66 | 26 | 5 × 10⁻⁶ | 9 | 1,000 | 13 |
| S180 | Soft Tissue | 33 | 8 | 2 × 10⁻³ | 1.5 | 2,000 | 9 |
| Siha | Cervix | 53 | 24 | 5 × 10⁻⁷ | 7 | 1,000 | 19 |
| U14 | Cervix | 46 | 6 | 3 × 10⁻² | 3 | 2,000 | 4 |

Throughout this document, various references are mentioned. All such references are incorporated herein by reference, including the references set forth in the following list:

### REFERENCE SIGNS LIST

1. Poland A, Knutson J C. 2,3,7,8-tetrachlorodibenzo-p-dioxin and related halogenated aromatic hydrocarbons: examination of the mechanism of toxicity. Annu Rev Pharmacol Toxicol. 1982; 22:517-554. doi:10.1146/annurev.pa.22.040182.002505.
2. Poellinger L. Mechanistic aspects—the dioxin (aryl hydrocarbon) receptor. Food Addit Contam. 2000; 17(4):261-6.
3. Bock K W, Kohle C. Ah receptor- and TCDD-mediated liver tumor promotion: clonal selection and expansion of cells evading growth arrest and apoptosis. Biochem Pharmacol. 2005; 69(10):1403-1408. doi:10.1016/j.bcp.2005.02.004.
4. Stevens E A, Mezrich J D, Bradfield C A. The aryl hydrocarbon receptor: a perspective on potential roles in the immune system. Immunology. 2009; 127(3):299-311. doi:10.1111/j.1365-2567.2009.03054.x.
5. Puga A, Tomlinson C R, Xia Y. Ah receptor signals cross-talk with multiple developmental pathways. Biochem Pharmacol. 2005; 69(2):199-207. doi:10.1016/j.bcp.2004.06.043.
6. Safe S, McDougal A. Mechanism of action and development of selective aryl hydrocarbon receptor modulators for treatment of hormone-dependent cancers (Review). Int J Oncol. 2002; 20(6): 1123-8.
7. DeLuca H F, Clagett-Dame M, Song J, Helfand S, Akhtar N. U.S. Pat. No. 7,419,992—Use of aryl hydrocarbon receptor ligand as a therapeutic intervention in angiogenesis-implicated disorders. 2008. Available at: http://patft.uspto.gov/netacgi/nph-Parser?Sect1=PTO2&Sect2=HITOFF&p=1&u=%2Fnetahtml %2FPTO %2Fsearch-bool.html&r=8&f=G&1=50&co1=AND&d=PTXT&s1=Jiasheng.INNM.&OS=IN/Jiasheng&RS=IN/Jiasheng. Accessed Jul. 19, 2013.
8. Dietrich C, Kaina B. The aryl hydrocarbon receptor (AhR) in the regulation of cell-cell contact and tumor growth. Carcinogenesis. 2010; 31(8):1319-1328. doi:10.1093/carcin/bgq028.
9. Song J, Clagett-Dame M, Peterson R E, et al. A ligand for the aryl hydrocarbon receptor isolated from lung. Proc Natl Acad Sci USA. 2002; 99(23):14694-9. doi:10.1073/pnas.232562899.
10. DeLuca H F, Song J, Clagett-Dame M, et al. U.S. Pat. No. 6,916,834—Preparations and use of an Ah receptor ligand, 2-(1′H-indole-3′-carbonyl)-thiazole-4-carboxylic acid methyl ester. 2005. Available at: http://patft.uspto.gov/netacgi/nph-Parser?Sect1=PTO2&Sect2=HITOFF&p=1&u=%2Fnetahtml%2FPTO%2Fsearch-bool.html&r=10&f=G&1=50&col=AND&d=PTXT&s1=Jiasheng.INNM.&OS=IN/Jiasheng&R S=IN/Jiasheng. Accessed Jul. 19, 2013.
11. Fritz W A, Lin T-M, Safe S, Moore R W, Peterson R E. The selective aryl hydrocarbon receptor modulator 6-methyl-1,3,8-trichlorodibenzofuran inhibits prostate tumor metastasis in TRAMP mice. Biochem Pharmacol. 2009; 77(7):1151-1160. doi:10.1016/j.bcp.2008.12.015.
12. McDougal A, Wilson C, Safe S Inhibition of 7,12-dimethylbenz[a]anthracene-induced rat mammary tumor growth by aryl hydrocarbon receptor agonists. Cancer Lett. 1997; 120(1):53-63.
13. Holcomb M, Safe S Inhibition of 7,12-dimethylbenzanthracene-induced rat mammary tumor growth by 2,3,7,8-tetrachlorodibenzo-p-dioxin. Cancer Lett. 1994; 82(1):43-47.
14. McDougal A, Wormke M, Calvin J, Safe S. Tamoxifen-induced antitumorigenic/antiestrogenic action synergized by a selective aryl hydrocarbon receptor modulator. Cancer Res. 2001; 61(10):3902-3907.
15. Gierthy J F, Bennett J A, Bradley L M, Cutler D S. Correlation of in vitro and in vivo growth suppression of MCF-7 human breast cancer by 2,3,7,8-tetrachlorodibenzo-p-dioxin. Cancer Res. 1993; 53(13):3149-3153.
16. Zhang S, Lei P, Liu X, et al. The aryl hydrocarbon receptor as a target for estrogen receptor-negative breast cancer chemotherapy. Endocr Relat Cancer. 2009; 16(3):835-844. doi:10.1677/ERC-09-0054.
17. Kawajiri K, Kobayashi Y, Ohtake F, et al. Aryl hydrocarbon receptor suppresses intestinal carcinogenesis in ApcMin/+ mice with natural ligands. Proc Natl Acad Sci USA. 2009; 106(32):13481-13486. doi:10.1073/pnas.0902132106.
18. O'Donnell E F, Kopparapu P R, Koch D C, et al. The aryl hydrocarbon receptor mediates leflunomide-induced growth inhibition of melanoma cells. Plos One. 2012; 7(7):e40926. doi:10.1371/journal.pone.0040926.
19. Simon T, Aylward L L, Kirman C R, Rowlands J C, Budinsky R A. Estimates of cancer potency of 2,3,7,8-tetrachlorodibenzo(p)dioxin using linear and nonlinear dose-response modeling and toxicokinetics. Toxicol Sci Off J Soc Toxicol. 2009; 112(2):490-506. doi:10.1093/toxsci/kfp232.
20. Ishida M, Mikami S, Kikuchi E, et al. Activation of the aryl hydrocarbon receptor pathway enhances cancer cell invasion by upregulating the MMP expression and is associated with poor prognosis in upper urinary tract urothelial cancer. Carcinogenesis. 2010; 31(2):287-295. doi:10.1093/carcin/bgp222.
21. Ray S, Swanson H I. Activation of the aryl hydrocarbon receptor by TCDD inhibits senescence: a tumor promoting event? Biochem Pharmacol. 2009; 77(4):681-688. doi:10.1016/j.bcp.2008.11.022.
22. Knerr S, Schrenk D. Carcinogenicity of 2,3,7,8-tetrachlorodibenzo-p-dioxin in experimental models. Mol Nutr Food Res. 2006; 50(10):897-907. doi:10.1002/mnfr.200600006.
23. Yu Z, Loehr C V, Fischer K A, et al. In utero exposure of mice to dibenzo[a,1]pyrene produces lymphoma in the offspring: role of the aryl hydrocarbon receptor. Cancer Res. 2006; 66(2):755-762. doi:10.1158/0008-5472.CAN-05-3390.
24. Song J. United States Patent Application: 0120214853—ITE for Cancer Intervention and Eradication. A1. Available at: http://appft1.uspto.gov/netacgi/nph-Parser?Sect1=PT02&Sect2=HITOFF&p=1&u=%2Fnetahtml %2FPTO %2Fsearch-bool.html&r=4&f=G&1=50&co1=AND&d=PG01&s1=Jiasheng.IN.&OS=IN/Jiasheng&RS=IN/Jiasheng. Accessed Jul. 19, 2013.
25. Quintana F J, Basso A S, Iglesias A H, et al. Control of T(reg) and T(H)17 cell differentiation by the aryl hydrocarbon receptor. Nature. 2008; 453(7191):65-71. doi:10.1038/nature06880.
26. Henry E C, Bemis J C, Henry O, Kende A S, Gasiewicz T A. A potential endogenous ligand for the aryl hydrocarbon receptor has potent agonist activity in vitro and in vivo. Arch Biochem Biophys. 2006; 450(1):67-77. doi:10.1016/j.abb.2006.02.008.
27. Brauze D, Widerak M, Cwykiel J, Szyfter K, Baer-Dubowska W. The effect of aryl hydrocarbon receptor ligands on the expression of AhR, AhRR, ARNT, Hiflalpha, CYP1A1 and NQO1 genes in rat liver. Toxicol Lett. 2006; 167(3):212-220. doi:10.1016/j.toxlet.2006.09.010.
28. Bermudez de Leon M, Gómez P, Elizondo G, Zatarain-Palacios R, Garcia-Sierra F, Cisneros B. Beta-naphthoflavone represses dystrophin Dp71 expression in hepatic cells. Biochim Biophys Acta. 2006; 1759(3-4):152-158. doi:10.1016/j.bbaexp.2006.03.005.
29. Okino S T, Pookot D, Basak S, Dahiya R. Toxic and chemopreventive ligands preferentially activate distinct aryl hydrocarbon receptor pathways: implications for cancer prevention. Cancer Prev Res Phila Pa. 2009; 2(3):251-256. doi:10.1158/1940-6207.CAPR-08-0146.
30. Morrow D, Qin C, Smith R, Safe S. Aryl hydrocarbon receptor-mediated inhibition of LNCaP prostate cancer cell growth and hormone-induced transactivation. J Steroid Biochem Mol. Biol. 2004; 88(1):27-36. doi:10.1016/j.jsbmb.2003.10.005.
31. Sanderson J T, Slobbe L, Lansbergen G W, Safe S, van den Berg M. 2,3,7,8-Tetrachlorodibenzo-p-dioxin and diindolylmethanes differentially induce cytochrome P450 1A1, 1B1, and 19 in H295R human adrenocortical carcinoma cells. Toxicol Sci Off J Soc Toxicol. 2001; 61(1):40-48.
32. John A R, Bramhall S R, Eggo M C. Antiangiogenic therapy and surgical practice. Br J. Surg. 2008; 95(3):281-293. doi:10.1002/bjs.6108.
33. Roukos D H, Tzakos A, Zografos G. Current concerns and challenges regarding tailored anti-angiogenic therapy in cancer. Expert Rev Anticancer Ther. 2009; 9(10):1413-1416. doi:10.1586/era.09.116.
34. English B C, Price D K, Figg W D. VEGF inhibition and metastasis: possible implications for antiangiogenic therapy. Cancer Biol Ther. 2009; 8(13):1214-1225.
35. Loges S, Mazzone M, Hohensinner P, Carmeliet P. Silencing or fueling metastasis with VEGF inhibitors: antiangiogenesis revisited. Cancer Cell. 2009; 15(3):167-170. doi:10.1016/j.ccr.2009.02.007.
36. Ebos J M L, Lee C R, Cruz-Munoz W, Bjarnason G A, Christensen J G, Kerbel R S. Accelerated metastasis after short-term treatment with a potent inhibitor of tumor angiogenesis. Cancer Cell. 2009; 15(3):232-239. doi:10.1016/j.ccr.2009.01.021.
37. Pàez-Ribes M, Allen E, Hudock J, et al. Antiangiogenic therapy elicits malignant progression of tumors to increased local invasion and distant metastasis. Cancer Cell. 2009; 15(3):220-231. doi:10.1016/j.ccr.2009.01.027.
38. Elizondo G, Fernandez-Salguero P, Sheikh M S, et al. Altered cell cycle control at the G(2)/M phases in aryl hydrocarbon receptor-null embryo fibroblast. Mol. Pharmacol. 2000; 57(5): 1056-63.
39. Puga A, Marlowe J, Barnes S, et al. Role of the aryl hydrocarbon receptor in cell cycle regulation. Toxicology. 2002; 181-182:171-7.
40. Marlowe J L, Knudsen E S, Schwemberger S, Puga A. The aryl hydrocarbon receptor displaces p300 from E2F-dependent promoters and represses S phase-specific gene expression. J Biol. Chem. 2004; 279(28):29013-22. doi:10.1074/jbc.M404315200.
41. Kajta M, Wójtowicz A K, Maekowiak M, Lason W. Aryl hydrocarbon receptor-mediated apoptosis of neuronal cells: a possible interaction with estrogen receptor signaling. Neuroscience. 2009; 158(2):811-822. doi:10.1016/j.neuroscience.2008.10.045.
42. Singh N P, Nagarkatti M, Nagarkatti P. Primary peripheral T cells become susceptible to 2,3,7,8-tetrachlorodibenzo-p-dioxin-mediated apoptosis in vitro upon activation and in the presence of dendritic cells. Mol. Pharmacol. 2008; 73(6):1722-1735. doi:10.1124/mol.107.043406.
43. Park K-T, Mitchell K A, Huang G, Elferink C J. The aryl hydrocarbon receptor predisposes hepatocytes to Fas-mediated apoptosis. Mol. Pharmacol. 2005; 67(3):612-22. doi:10.1124/mol.104.005223.
44. Jux B, Kadow S, Esser C. Langerhans cell maturation and contact hypersensitivity are impaired in aryl hydrocarbon receptor-null mice. J Immunol Baltim Md 1950. 2009; 182(11):6709-6717. doi:10.4049/jimmunol.0713344.
45. Sutter C H, Yin H, Li Y, et al. EGF receptor signaling blocks aryl hydrocarbon receptor-mediated transcription and cell differentiation in human epidermal keratinocytes. Proc Natl Acad Sci USA. 2009; 106(11):4266-4271. doi:10.1073/pnas.0900874106.
46. Hall J M, Barhoover M A, Kazmin D, McDonnell D P, Greenlee W F, Thomas R S. Activation of the Aryl-Hydrocarbon Receptor Inhibits Invasive and Metastatic Features of Human Breast Cancer Cells and Promotes Breast Cancer Cell Differentiation. Mol Endocrinol Baltim Md. 2009. doi:10.1210/me.2009-0346.
47. Oenga G N, Spink D C, Carpenter D O. TCDD and PCBs inhibit breast cancer cell proliferation in vitro. Toxicol Vitro Int J Publ Assoc Bibra. 2004; 18(6):811-9. doi:10.1016/j.tiv.2004.04.004.
48. Jana N R, Sarkar S, Ishizuka M, Yonemoto J, Tohyama C, Sone H. Cross-talk between 2,3,7,8-tetrachlorodibenzo-p-dioxin and testosterone signal transduction pathways in LNCaP prostate cancer cells. Biochem Biophys Res Commun. 1999; 256(3):462-8. doi:10.1006/bbrc.1999.0367.
49. Morrow D, Qin C, Smith R, Safe S. Aryl hydrocarbon receptor-mediated inhibition of LNCaP prostate cancer cell growth and hormone-induced transactivation. J Steroid Biochem Mol. Biol. 2004; 88(1):27-36. doi:10.1016/j.jsbmb.2003.10.005.
50. Veldhoen M, Hirota K, Westendorf A M, et al. The aryl hydrocarbon receptor links TH17-cell-mediated autoimmunity to environmental toxins. Nature. 2008; 453(7191):106-109. doi:10.1038/nature06881.
51. Koliopanos A, Kleeff J, Xiao Y, et al. Increased arylhydrocarbon receptor expression offers a potential therapeutic target for pancreatic cancer. Oncogene. 2002; 21(39):6059-6070. doi:10.1038/sj.one.1205633.
52. Kashani M, Steiner G, Haitel A, et al. Expression of the aryl hydrocarbon receptor (AhR) and the aryl hydrocarbon receptor nuclear translocator (ARNT) in fetal, benign hyperplastic, and malignant prostate. Prostate. 1998; 37(2):98-108.
53. Gluschnaider U, Hidas G, Cojocaru G, Yutkin V, Ben-Neriah Y, Pikarsky E. beta-TrCP inhibition reduces prostate cancer cell growth via upregulation of the aryl hydrocarbon receptor. Plos One. 2010; 5(2):e9060. doi:10.1371/journal.pone.0009060.
54. Peng T-L, Chen J, Mao W, et al. Potential therapeutic significance of increased expression of aryl hydrocarbon receptor in human gastric cancer. World J Gastroenterol Wjg. 2009; 15(14):1719-1729.
55. Liu Z, Wu X, Zhang F, et al. AhR expression is increased in hepatocellular carcinoma. J Mol Histol. 2013. doi:10.1007/s10735-013-9495-6.
56. Lin P, Chang H, Tsai W-T, et al. Overexpression of aryl hydrocarbon receptor in human lung carcinomas. Toxicol Pathol. 2003; 31(1):22-30.
57. Zhang J, Zong H, Li S, Zhang D, Zhang L, Xia Q. Activation of aryl hydrocarbon receptor suppresses invasion of esophageal squamous cell carcinoma cell lines. Tumori. 2012; 98(1):152-157. doi:10.1700/1053.11514.

It will be understood that various details of the presently disclosed subject matter can be changed without departing from the scope of the subject matter disclosed herein. Furthermore, the foregoing description is for the purpose of illustration only, and not for the purpose of limitation.

1. A method of cancer treatment comprising administering a therapeutically effective amount of 2-1′H-indole-3′-carbonyl)-thiazole-4-carboxylic acid methyl ester or one of its structural analogs, wherein the cancer is selected from a group consisting of skin, colorectal, stomach, pancreatic, kidney, bladder, soft tissue, and cervical cancer, wherein the said structural analog of 2-1′H-indole-3′-carbonyl)-thiazole-4-carboxylic acid methyl ester has the formula: 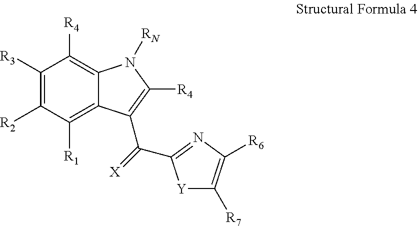
wherein
X and Y, independently, can be either O (oxygen) or S (sulfur);
R_{N} can be selected from hydrogen, halo, cyano, formyl, alkyl, haloalkyl, alkenyl, alkynyl, alkanoyl, haloalkanoyl, or a nitrogen protective group;
R₁, R₂, R₃, R₄, and R₅ can be independently selected from hydrogen, halo, hydroxy (—OH), thiol (—SH), cyano (—CN), formyl (—CHO), alkyl, haloalkyl, alkenyl, alkynyl, amino, nitro (—NO₂), alkoxy, haloalkoxy, thioalkoxy, alkanoyl, haloalkanoyl, or carbonyloxy;
R₆ and R₇, can be independently selected from hydrogen, halo, hydroxy, thiol, cyano, formyl, alkyl, haloalkyl, alkenyl, alkynyl, amino, nitro, alkoxy, haloalkoxy, or thioalkoxy; or
R₆ and R₇, independently, can be: 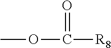
wherein R₈ can be selected from hydrogen, halo, cyano, alkyl, haloalkyl, alkenyl, or alkynyl; or
R₆ and R₇, independently, can be: 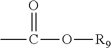
wherein R₉ can be selected from hydrogen, halo, alkyl, haloalkyl, alkenyl, or alkynyl; or
R₆ and R₇, independently, can be: 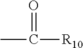
wherein R₁₀ can be selected from hydrogen, halo, hydroxy, thiol, cyano, alkyl, haloalkyl, alkenyl, alkynyl, amino, or nitro; or
R₆ and R₇, independently, can also be: 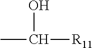
wherein R₁₁ can be selected from hydrogen, halo, alkyl, haloalkyl, alkenyl, or alkynyl.

2. The method of claim 1, wherein the structure of said 2-(1′H-indole-3′-carbonyl)-thiazole-4-carboxylic acid methyl ester is represented by the following structural formula:

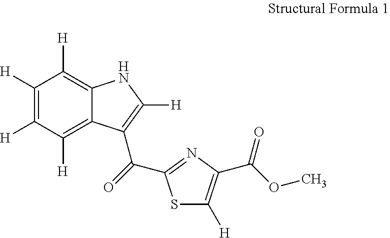 [SMILES: COC(=O)c1csc(C(=O)c2c[nH]c3ccccc23)n1]

3. The method of claim 1, wherein the structural analog of 2-1′H-indole-3′-carbonyl)-thiazole-4-carboxylic acid methyl ester is represented by the following structural formula:

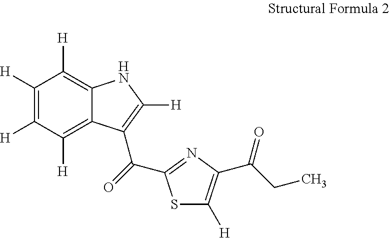 [SMILES: CCC(=O)c1csc(C(=O)c2c[nH]c3ccccc23)n1]

4. The method of claim 1, wherein the structural analog of 2-1′H-indole-3′-carbonyl)-thiazole-4-carboxylic acid methyl ester is represented by the following structural formula:

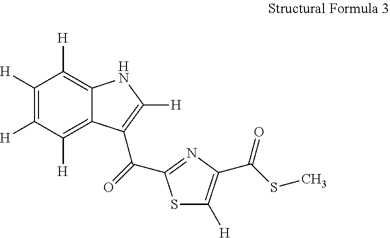 [SMILES: CSC(=O)c1csc(C(=O)c2c[nH]c3ccccc23)n1]

5. The method of claim 1, wherein the 2-(1′H-indole-3′-carbonyl)-thiazole-4-carboxylic acid methyl ester or one of its structural analogs is combined with one or more pharmaceutically acceptable carriers to assist its administration to the subject.

6. The method of claim 1, wherein the step of administering the 2-(1′H-indole-3′-carbonyl)-thiazole-4-carboxylic acid methyl ester or one of its structural analogs is selected from a group consisting of topical, enteral, and parenteral application.

7. The method of claim 1, further comprising administering a dose of water to the subject to reduce complication of administering the 2-1′H-indole-3′-carbonyl)-thiazole-4-carboxylic acid methyl ester or one of its structural analogs.

8. The method of claim 7, wherein the dose of water is administered by oral injection and is in addition to normal water uptake by the subject.

9. The method of claim 7, wherein the complication is feces hardening.

10. The method of claim 1, wherein the 2-1′H-indole-3′-carbonyl)-thiazole-4-carboxylic acid methyl ester or one of its structural analogs is administered together with one or more other cancer therapeutic agents to the subject.

11. The method of claim 1, wherein a maintenance dosing of the 2-1′H-indole-3′-carbonyl)-thiazole-4-carboxylic acid methyl ester or one of its structural analogs is provided after the subject is free of cancer to ensure cancer eradication.

12. The method of claim 1, wherein the subject is a mammal.

13. The method of claim 12, wherein the mammal is a human.

14. The method of claim 1, wherein the subject is administered with the 2-1′H-indole-3′-carbonyl)-thiazole-4-carboxylic acid methyl ester or one of its structural analogs once or twice daily.
